(19) United States
(12) Patent Application Publication
(10) Pub. No.: US 2026/0109959 A1
(43) Pub. Date: Apr. 23, 2026

(54) METHODS OF TREATING PULMONARY SARCOIDOSIS WITH DNA NUCLEASES

(71) Applicants: Brian William Davies, Newport Beach, CA (US); Daniel S. Cartwright, Houston, TX (US); Ira Kalfus, New York, NY (US)

(72) Inventors: Brian William Davies, Newport Beach, CA (US); Daniel S. Cartwright, Houston, TX (US); Ira Kalfus, New York, NY (US)

(21) Appl. No.: 19/031,963

(22) Filed: Jan. 18, 2025

(51) Int. Cl. C12N 9/22; A61K 9/00; A61K 38/00; A61P 11/00; A61P 37/06

(52) U.S. Cl. CPC C12N 9/22; A61K 9/0073; A61P 11/00; A61P 37/06; A61K 38/00; C12Y 301/21001

(57) ABSTRACT

The present invention is directed at methods for treating pulmonary sarcoidosis comprising the therapeutic administration of human recombinant DNase1 or other DNA nuclease enzymes directly inhaled into the lungs. DNA nucleases are used herein for the breakdown of excessive lung microbial, mitochondrial, and other free DNA. Such excessive DNA enters dendritic cells and macrophages by way of toll receptor 2 (TLR2) to stimulate toll receptor 9 (TLR9). The TLR9 receptor initiates the inflammation cascade in sarcoidosis with release of pro-inflammatory cytokines and resultant inflammatory cascade resulting in fibrosis and granuloma formations. Therapeutically dosed DNA nucleases such as recombinant human DNase 1 inhaled into sarcoidosis lungs reduce excessive DNA levels, and the associated stimulation of cytokines, inflammation, and granulomas. DNase 1 therapy would reduce inflammation and treat the cause of the pulmonary sarcoidosis. DNase 1 would be used to reduce the dose of steroid therapies and their concomitant serious complications.

FIELD OF THE INVENTION The present invention is directed to methods for treating pulmonary sarcoidosis comprising the administration of DNase 1-like nucleases for the breakdown of excessive lung microbial, mitochondrial and other free DNA. Excess DNA enters dendritic cells and macrophages to stimulate toll receptor 9 (TLR9) and initiate the inflammation cascade associated with pulmonary sarcoidosis. Therapeutic DNase 1 in sarcoidosis lungs [as shown in animal model of sarcoidosis] reduces DNA levels and associated stimulation of pro-inflammatory cytokines, inflammation and granulomas.

BACKGROUND OF THE INVENTION Deoxyribonuclease (DNase I) is a natural occurring enzyme that selectively hydrolyzes single and double stranded DNA. DNase I, as a member of the DNase I family it cleaves DNA to form two products with 5′ phospho and 3′ hydroxy ends. DNases I are active at a pH of 6.5-8 and need bivalent magnesium and calcium cations to be active. Starting from 1995, the next three members of the DNase I family were described—DNase1L1, DNase1L2 and DNase1L3. These nucleases are also known as DNase I-like nucleases, because their amino acid sequences and catalytic properties are like DNase I.

Clinically a recombinant human DNase I, dornase alfa, is produced in Chinese Hamster Ovary (CHO) cells and marketed as Pulmozyme®. It is FDA approved for the treatment of patients with cystic fibrosis. In patients with cystic fibrosis this product acts primarily as a mucolytic. Additionally, a second recombinant human DNase I, Alidornase Alpha, expressed in plant cells with a chemical modification of the enzyme that imparts improved resistance to actin inhibition, thereby giving a longer duration of nuclease activity. This drug is manufactured by Protalix Biotherapeutics in Israel.

DNase 1 is an endonuclease found in eukaryotic cells that cleaves phosphodiester linkages in DNA. Included is single stranded DNA, double stranded DNA, and chromatin. DNase I is an enzyme that plays a crucial role in DNA metabolism. During programmed cell death (apoptosis), DNase 1 is involved in the fragmentation of DNA, helping to dismantle the cells genetic material. The composition of Pulmozyme® by Roche/Genentech is a human recombinant DNA (“rhDNase”) and is a glycoprotein containing 260 amino acids with a molecular weight of 37,000 Daltons. The amino acid sequence is identical to native human DNase 1. There may be other modifications of DNase 1 that work as longer acting endonucleases which may be used in the future.

Evidence suggests that DNase 1 also acts on bacterial DNA to reduce recurrent pseudomonas infections from bacteria found in a biofilm within a patient's lung (1). Additionally, the amount of P. acnes DNA in BAL cells from patients with sarcoidosis was significantly higher than that in BAL cells from patients with other pulmonary diseases. P. acnes may be involved in the pathogenesis of sarcoidosis. Observations suggest that P. acnes may reside indigenously and proliferate in the lower respiratory tract. Pulmonary granulomas in patients with sarcoidosis may result from delayed hypersensitivity to P. acnes, which may play a central role in the antigen-driven immune response in the lungs of patients with sarcoidosis. (Quantitative Analysis of Propionibacterial DNA in Bronchoalveolar Lavage Cells from Patients with Sarcoidosis (2).

Because of their clinical and immunological features, mycobacteria were thought to be the most likely causative infective agents of sarcoidosis. Some investigators detected mycobacterial DNA by using PCR in tissue samples from patients with pulmonary sarcoidosis. Using PCR, Gazouli showed the existence of P. granulosum (43.8%) and M. tuberculosis (71.7%) in tissue samples (lung and lymph nodes) from patients with sarcoidosis (3).

Sarcoidosis is a systemic granulomatous disease characterized by a T-helper-1 response with accumulation of CD4+ lymphocytes and activated macrophages in the lungs and affected organs, resulting in characteristic granuloma formation in genetically pre-disposed individuals (4) (5). Sarcoidosis most commonly affects young adults of both sexes, although studies have reported more cases in females. The incidence is highest for individuals younger than 40 and peaks in the age-group from 20 to 29 years; a second peak is observed for women over 50 (6).

Sarcoidosis is a chronic inflammatory condition characterized by granulomatous inflammation of unknown origin (7). Both pulmonary and extrapulmonary symptoms and signs may be present as clinically recognizable syndromic patterns, but unusual presentations may be challenging (8). A dry cough is common, but a productive cough [excessive mucus] suggests an alternative diagnosis (9) Sarcoidosis occurs throughout the world in all races with an average incidence of 16.5 per 100,000 in men and 19 per 100,000 in women. The disease is most common in Northern European countries and the highest annual incidence of 60 per 100,000 is found in Sweden and Iceland. In the United Kingdom the prevalence is 16 per 100,000. In the United States, sarcoidosis is more common in people of African descent than Caucasians, with annual incidences reported as 35.5 and 10.9 per 100,000, respectively (10).

The etiology of this disease is still unclear. (11) It is hypothesized that one or more unidentified antigens induce an immune response mediated by alveolar macrophages and lymphocytes. Due to similarities with granulomas formed during mycobacterial infections, it is hypothesized that DNA containing complexes originating from a variety of bacteria may be one of these potential antigens. (12) (13) (14).

There is evidence of the presence of microbial DNA from mycobacteria, propionibacteria and borrelia in sarcoidosis tissues suggesting they may participate in sarcoidosis granuloma formation (15). Bacterial amyloid curli may act as a carrier for such DNA to elicit an autoimmune response. (16) (17)

Serum amyloid A is derived from the host and may participate in the granuloma formation by regulating inflammation through Toll-like receptor-2 (TLR2) (18). Serum amyloid A is localized to macrophages and giant cells within sarcoidosis granulomas but correlated with CD3 (+) lymphocytes, linking expression to local Th1 responses (18). Serum amyloid A activated NF-kappaβ in TLR2-expressing human cell lines, regulated experimental Th1-mediated granulomatous inflammation through interferon (IFN)-gamma, tumor necrosis factor, interleukin (IL)-10, and TLR-2; and stimulated production of tumor necrosis factor, IL-10, and IL-18 in lung cells from patients with sarcoidosis, effects inhibited by blocking TLR-2 (18).

Pathology—The granuloma in pulmonary sarcoidosis is characterized by a core of monocyte-derived epithelioid histiocytes and multinucleate giant cells with interspersed CD4+T lymphocytes. A minority of cells in or near the granuloma are CD8+T lymphocytes, fibroblasts, regulatory T cells, and B lymphocytes. The T-cell response is biased toward a Th1 phenotype, with important roles for IFN-γ and IL-12 (19). Increased expression of key inflammatory mediators, tumor necrosis factor (TNF), IFN-γ, IL-2, IL-10, IL-12, IL-18 and transcription factor STAT-1, is indicative of a Th1-mediated immune response. (20)

More recently, studies have found upregulation of Th17 immune responses producing IL17 and Th17.1 responses expressing INF gamma in sarcoidosis (21) (22). Several studies indicate an expansion of regulatory T cells but with reduced functional capabilities that may be ineffective in suppressing the local exaggerated T cell mediated inflammatory tissue responses (23) (24). Sarcoidosis has paradoxical effects on inflammatory processes, characterized by increased macrophage and CD4 helper T-cell activation, resulting in accelerated inflammation, but immune response to antigen challenges such as tuberculin is suppressed in the peripheral blood and skin. This response may be mediated by upregulation of the immune checkpoint inhibitor Programmed Death-1 (PD-1) on peripheral blood CD4 T cells in sarcoidosis patients (25).

Clinical Presentation-Patients with pulmonary sarcoidosis commonly present with systemic symptoms, including fatigue, swollen lymph nodes and weight loss. Most patients with sarcoidosis experience lung problems, which may include persistent dry cough, shortness of breath, wheezing and chest pain. Some patients are asymptomatic, but when symptoms develop, findings usually depend on the extent of specific organ involvement (26). The lungs are affected in more than 90% of patients with sarcoidosis, thus abnormal pulmonary function is characteristic for many patients (27). Overall, about 50% of sarcoidosis patients develop permanent pulmonary abnormalities, and 5 to 15% have progressive fibrosis of lung parenchyma. Sarcoidosis of the lung is primarily an interstitial disease in which the inflammatory process involves the alveoli, small bronchi, and small blood vessels (28). At least 5% of people suffer from pulmonary arterial hypertension (29).

Diagnosis-Diagnosis relies on three criteria: (1) a compatible clinical and radiologic presentation, (2) pathologic evidence of noncaseating granulomas, and (3) exclusion of other diseases with similar findings, such as infections or malignancy. Laboratory studies are also recommended and include a peripheral white blood cell count, serum chemistries including calcium and creatinine levels, liver function tests, urinalysis, and electrocardiogram. Flexible bronchoscopy with biopsies has shown high diagnostic yields for pulmonary sarcoidosis (30). The historic staging system for pulmonary sarcoidosis based on chest radiographic findings at initial diagnosis (31).

### Radiologic Staging of Pulmonary Sarcoidosis

| Stage | Chest Radiography Results |
|---|---|
| 0 | No adenopathy or infiltrates |
| 1 | Hilar and mediastinal adenopathy alone |
| 2 | Adenopathy and pulmonary infiltrates |
| 3 | Pulmonary infiltrates alone |
| ′4 | Pulmonary fibrosis |

Chest radiography is useful in following the clinical course of patients on therapy (32).

Clinical laboratory evaluation, serum biomarkers and bronchoalveolar lavage are also used to rule out infectious and oncological causes that may mimic sarcoidosis and further support the diagnosis of pulmonary sarcoidosis (33). In addition, pulmonary function testing is useful both in making the diagnosis and in evaluating response to therapy (34). Several biomarkers have been proposed that may help with diagnosis and guide a response to therapy. Though there are no currently accepted biomarkers that have widespread acceptance or consensus for either a diagnosis or guide to therapy (35).

Current Treatments and Standard of Care—The natural course of the disease and prognosis are variable from asymptomatic, spontaneous remission over many months to progressive disease requiring treatment (34). Corticosteroids are recommended as first-line therapy, but an optimal regimen and duration of treatment is not well established. Once treatment is initiated, tapering therapy can be problematic with recurrent inflammation and functional declines. Treatment may differ based on severity of disease, extrapulmonary involvement, as well as physician and patient preferences. Corticosteroid use is effective as an initial therapy but is associated with significant side effects which can impair patients' quality of life (36). Many of the toxicities of corticosteroids are cumulative side effects such as weight gain, diabetes, osteoporosis, cataracts, and skin fragility (36).

Steroid sparing strategies involve immunomodulating drugs that have their own individual drawbacks and toxicities. Agents in common use include methotrexate, leflunomide, azathioprine, mycophenolate, hydroxychloroquine, and infliximab (37). Methotrexate is often considered as an initial steroid sparing therapy in those who do not have contraindications to its use. Long-term treatment with methotrexate is associated with liver damage in about 10% of people and hence may be a significant concern in people with liver involvement and require regular liver function test monitoring (38).

Methotrexate can also lead to pulmonary toxicity, although this is uncommon and more commonly it can confound the leukopenia caused by sarcoidosis (38). Due to these safety concerns it is recommended that methotrexate is used with folic acid in order to reduce toxicity (38). Azathioprine treatment less frequently can lead to liver damage but carries a higher rate of infectious complications (39) (40). Leflunomide is sometimes being used as a replacement for methotrexate, possibly due to its purportedly lower rate of pulmonary toxicity (40). Mycophenolic acid has been used successfully for pulmonary sarcoidosis (41).

The anti-TNF agent, infliximab has shown marginal benefit in pulmonary sarcoidosis but may be more effective in non-pulmonary sarcoidosis (42) (43). The anti-TNF agent adalimumab has also been used as a steroid sparing therapy supported by small anecdotal case series.

Basic science research that supports the Inventors' MOA. The following describes the mechanism of action (MoA) of DNase1 in (“prokaryotic DNA”) or (“pDNA/TLR9”) dependent inflammatory cascade with its chronic resultant fibrotic disease process. Based on this MoA, there is the expectation that DNAse1 [and its variants] or other DNA nucleases will reduce “pDNA” pro-inflammatory and pro-fibrotic mediators. As a result of relative reduction in total “pDNA” available to this stimulation there will be a reduction in the progression of inflammation and fibrosis. Logically this will reduce or resolve pre-existing inflammation. This use of DNase 1 may resolve pre-existing fibrosis in animal models or patients with fibrotic diseases. Details of this proposed mechanism of action will be fully described below. At a molecular level, DNAse1 cleaves DNA into small oligonucleotide sequences. If the DNA is eukaryotic [human-complex cell structure and nucleus] in origin and/or prokaryotic in origin, this results in a reduction in viscosity of the mucus in the medium in which the DNA is found. This is the MoA of the current approved use of DNAse1 in cystic fibrosis. Therefore, Sarcoidosis has a dry cough and minimal mucous production compared to cystic fibrosis.

If the DNA is prokaryotic in origin (pDNA), and therefore has hypomethylated CpG sequences, the pDNA, once cleaved by DNAse1 cannot upregulate TLR9. Consequently. TLR9 does not upregulate pro-inflammatory and pro-fibrotic mediators. This may affect the disease progression of pulmonary sarcoidosis and related conditions. In concept this may reduce or eliminate the needs for steroids, a major goal in the clinical setting where the complications of taking steroids are severe.

The two sources of pDNA in a patient are bacterial DNA (btDNA) and mitochondrial DNA (mtDNA), Mitochondria are the descendants of bacteria internalized into a eukaryotic cell (44). When bacteria or mitochondria are damaged, free btDNA and mtDNA are released. The causes of these events include, a.) Normal bacterial cell death; b.) Bacterial death due to host immune responses to infection (e.g. pediatric sepsis) (45). c.) Patient cell damage due to physical trauma (e.g. ventilation) (46), or chemical trauma (e.g. paraquot or bleomycin) (47) and d.) Aging results in increases in mtDNA (48). All of these events lead to increased btDNA and/or increased mtDNA levels.

Increased mtDNA levels have been implicated in many diseases (49) (50) and animal models of disease (e.g. sterile and infectious SIRS in non-human primates) (51). In a study examining mtDNA in rheumatoid arthritis patients, PCR-amplifiable mtDNA was detected in 38 of 54 (70%) of the samples from rheumatoid arthritis patients and in 0 of 17 (0%) of the control patients (52).

Measurement of mtDNA copy number demonstrated that lepromatous leprosy patients had a significantly higher mtDNA content than control patients (p=0.008) (53). Increased pDNA levels result in increased TLR9 activation, which increases pro inflammatory and pro fibrotic mediators. This, in turn, results in the condition or disease noted, such as cardiovascular disease, granulosis with polyangitis. (55)

Circulating mitochondrial DNA in serum of patients with granulomatosis with polyangiitis (56), erythema nodosum leprosum (ENL) when compared with non-reactional lepromatous patients (57), and others (58). In animal models employing lipopolysaccharide (LPS) to mimic sepsis. LPS-induced mtDNA release occurs in a TLR4-dependent manner, and mtDNA causes acute lung injury and systemic inflammation in a TLR9-dependent and TLR4-independent manner (59).

In animal models using bleomycin or paraquot to induce inflammation and lung damage because of subsequent elevated mtDNA levels, systemically delivered DNAse1 delivered prophylactically reduces mtDNA levels and subsequently reduces inflammation and fibrosis (60). In human umbilical vein endothelial cells, lysosomal DNases protect cells against inflammation from mtDNA damage induced by ox-LDL (61). Small interfering RNA (siRNA) knockdown of DNase I amplifies the mtDNA/TLR9 mediated inflammatory response. It is likely that the specific condition is related to the specific trigger event that leads to bacterial or mitochondrial damage.

Several conditions and infections have been implicated in sarcoidosis, including bacterial infections such as Propionibacterium and Mycobacterium (62), and these have been shown to result in upregulation of TLR9 (63). Streptococcus pneumonia exposed to human alveolar epithelial cells in vitro causes the release of mtDNA (64).

Mitochondrial DNA has also been used to directly induce lung damage in animal models via TLR9 activation (65). In lung damage murine models induced by mtDNA, treatment with systemically delivered DNAse1 reduces lung epithelial permeability damage (66).

Several animal models have been developed that reflect parts of the sarcoidosis pathology (67). Investigations have revealed that bacteria upregulate TLR9 in HEK-TLR9 cells, including (from high to low upregulation), P. aeruginosa, M. tuberculosis, K. pneumoniae, and P acnes (68). This has led to animal models being developed using Propionibacterium to induce inflammation, granulomas, and fibrosis (69) (70), demonstrate TLR9 upregulation (71), and demonstrate the lack of ability of Propionibacterium to induce inflammation and granulomatosis in TLR9−/− mutant murine models (72) (73),

DNA from M. tuberculosis induces pro-inflammatory cytokine responses through a TLR9 dependent pathway in murine models of TLR9+/+ and TLR9−/− mutants. (74) Similar animal models have been developed using P aeruginosa. In murine models employ P aeruginosa, which upregulates TLR9 to a greater degree than M. tuberculosis and P acne (75). Treatment with systemically delivered DNase 1 reduces lung epithelial permeability damage (76).

Granulomatosis is suppressed in other infectious disease models of TLR9 −/− mutant mice including Stachybotrys chartarumiou (a fungus) induced hypersensitivity pneumonitis (77). It has been demonstrated that TLR9 is expressed in alveolar macrophages from patients with sarcoidosis with chest X-ray types I and II. These TLR9 receptors are fully active, as these cells react to the TLR9 ligand CpG. TLR9 was found to contribute to the immunopathogenesis of sarcoidosis via the induction of C-X-C motif chemokine 10 release in the alveolar macrophages (78). In the study in question, upregulation in TLR9 mRNA expression was detected in 14 patients with pulmonary sarcoidosis (p=0.05), when compared to 10 control patients (79).

In animal models of P. acne in mice that are TLR9−/−, which genetically reduces TLR9 levels, lung inflammation and granulosis are inhibited as compared to control wild type mice (80). In animal models in mice that are either TLR9−/−, or TLR9+/+, TLR9−/− mice challenged with mtDNA had no inflammatory response, while TLR9+/+ mice had significant upregulation of inflammatory mediators (81). The cleaving of pDNA by systemically delivered DNAse1 may therefore result in the amelioration of a pDNA/TLR9 mediated condition, including pulmonary sarcoidosis, by reducing the upregulation of TLR9.

Animal models where DNAse1 is administered intravenously or intraperitoneally demonstrate a preferred route of administration that addresses the MoA of DNAse1 used in pDNA/TLR9 dependent inflammation and fibrosis (82) (83) (84).

Additional patient data demonstrating the role of TLR9 upregulation in sarcoidosis supports the MoA for the use of DNAse1 in patients with sarcoidosis (85) (86). The Inventors believe it is rational for treating pulmonary sarcoidosis patients with DNAse1 or other variant nucleases that work to break down DNA.

### PRIOR ART

US20160067317A1

### Methods of Treating Pulmonary Sarcoidosis

Methods of treating pulmonary sarcoidosis are described herein. Patients in need of treatment for pulmonary sarcoidosis are administered a therapeutically effective amount of a mucolytic agent such as DNase I. In some embodiments, the DNase I is a recombinant human DNase I such as dornase alfa. Granted Aug. 2, 2016, Mr. Barry Burns. Status Expired—Fee related.

Novelty of current Inventors' patent: Inventors hereinafter address the novelty of the Inventors' patent from that of the prior art. First, the previous patent was authored by a PhD respiratory therapist, who used it for a mucolytic effect, such as cystic fibrosis, and combined it with other mucolytic agents. Cystic fibrosis (CF) causes increased thick mucus due to a genetic mutation in the cystic fibrosis transmembrane conductance regulator (CFTR) gene. This mutation affects the CFTR protein, which is responsible for regulating the movement of chloride ions across cell membranes. Normally, the CFTR protein helps transport chloride ions out of cells. Chloride is a component of salt, and its movement helps draw water to the cell surface, keeping mucus thin and slippery. In people with CF, the CFTR protein is defective or absent, leading to reduced chloride transport. Without sufficient chloride, water is not drawn to the cell surface, resulting in dehydrated, thick, and sticky mucus. Managing this thick mucus is a key part of CF treatment, involving therapies to hydrate and clear mucus from the lungs and other affected areas (101).

Unlike cystic fibrosis, pulmonary sarcoidosis patients have a persistent dry cough. In one series of 36 consecutive exacerbations of pulmonary sarcoidosis (defined as worsening pulmonary symptoms, worsening spirometry, and no clinical evidence of an alternative cause of pulmonary worsening other than pulmonary sarcoidosis) cough was present in 88 percent of the patients. It was more common than any other pulmonary symptom including dyspnea, wheeze, and chest pain (102). Therefore, cough is a sensitive, although not a specific, finding of active pulmonary sarcoidosis. The dry cough is often chronic in sarcoidosis, with more than one half of pulmonary sarcoidosis patients experiencing a cough of greater than 8 weeks duration. Additionally, a significant percentage experienced dry irritative cough for more than one year (103). In dry coughs there is little mucous formed.

Treatment with corticosteroids should be considered for patients with significant symptomatic or progressive stage II or III pulmonary disease or serious extrapulmonary disease. A meta-analysis identified 13 studies with 1,066 patients treated with systemic corticosteroids for six (6) to twenty-four (24) months. The authors found improvements in chest radiography findings, and limited data showed improvements in a global score incorporating symptoms and lung function in patients with stage II or III disease (104). Steroids would have an overall anti-inflammatory effect of resolving symptoms. The use of DNase 1 works [unlike steroids] to reduce inflammation at the source and treat the disease. By reducing the DNA available to enter the dendritic cell or macrophage and thus less intercellular DNA to stimulate TLR9. Therefore, the Inventors MOA would work to reduce the need for steroids and lessen the frequency of exacerbations and resultant chronic fibrotic damage to the lungs.

The expired priority patent cited Mr. Barry Burns' friend, a single black female patient with a 10-year history of Sarcoidosis. The Inventor, Mr. Burn's [PhD physiotherapist] was not the treating physician. No other scientific research was contemplated on the mechanism of action. All work supported the same therapy given to cystic fibrosis patients. He also included other mucolytic agents in the expired patent.

Most patients with sarcoidosis have a dry non-productive cough as opposed to a thick mucus cough as in cystic fibrosis. Researchers identified new regulatory pathways for mucus production (105). Mucus can be induced by Th2 and non-Th2 inflammatory responses in the lung, both of which are inhibited by IFN-γ. The blockade of eosinophilia and mucus production by IFN-γ likely occurs through different inhibitory pathways that are activated downstream of Th2 cytokine secretion and require IFN-γ signaling in tissue of recipient mice (105).

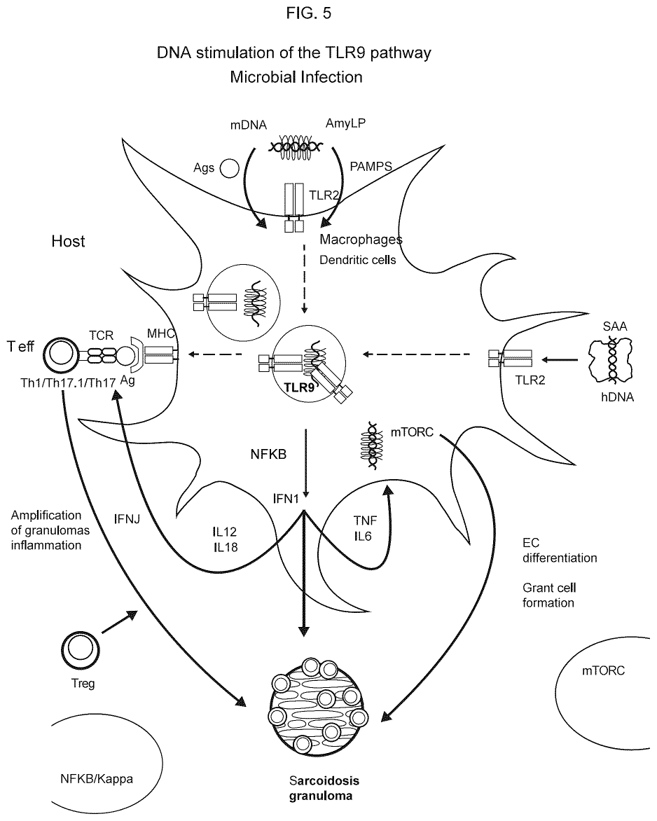

Non-Obviousness: The prior art would anticipate the patient to have a productive cough with mucus to work as a mucolytic. DNase I is commonly prescribed to patients with cystic fibrosis. It helps break down the thick mucus in the lungs by cleaving extracellular DNA, making it easier for patients to breathe. The current invention using DNase 1 in sarcoidosis works by reducing inflammation caused by networks of microbial and mitochondrial DNA. It dramatically decreases inflammation. It has little to do with the mucolytic effects. It is not obvious for someone in the field and has taken the Inventors seven years to work out this mechanism of action. See detailed description of the Inventors' MOA. (FIG. 5)

Inventors, sponsored animal proof of concept study number ISPT 20181114-1 (90). DNase I effects in Propionibacterium Acnes Mice Induced Pulmonary Sarcoidosis. The Inventors moved next to an approved animal model for sarcoidosis to prove the DNase 1 benefits in sarcoidosis. A third party CRO performed the study using Pulmozyme by Genentech/Roche. The purpose of this study was to evaluate the efficacy of DNase I to reduce P. Acnes-induced pulmonary sarcoidosis. This experiment was designed to evaluate (control or DNase 1 treated) lung cytokines, inflammation and fibrosis following treatment in sarcoidosis induced mice presenting with sarcoidosis disease.

An FDA recommended animal model of sarcoidosis was used to evaluate the efficacy of inhaled DNase I in reducing P. Acnes-induced pulmonary sarcoidosis. (87). Proprionic bacterium was used per protocol. Propionibacterium acnes was suspended with 0.5 mL tryptic soy broth supplemented with 5% defibrinated sheep's blood. The suspension was diluted in the same broth and P. acnes was growth at 37° C. in anaerobic conditions until enough number of bacteria was obtained. P. acnes was weighed, and the appropriate volume of sterile PBS was added to make up a concentration of 2 mg/ml or 10 mg/ml. The PA suspensions were heat-killed (80° C. fo30 minutes), aliquoted and stored at −20° C. A total of 16 mice were used for this study (2 groups, n=8). All C57BL/6 mice were intraperitoneally sensitized with 0.5 mg heat-killed PA (0.25 ml of 2 mg/ml suspension). Two weeks and four weeks following the intraperitoneal injection, the PA-sensitized mice were challenged, by instillation, with 0.5 mg of heat-killed PA (50 μL of 10 mg/mL suspension), 6 weeks after IP injection, lung tissues, as well as bronchoalveolar lavage fluid (BALF) were collected for further analysis (lung histopathology, hydroxyproline and collagen concentration, cytokine levels). Two weeks following the initial Intraperitoneal injection a second injection was performed to allow sensitization of the mice. On Day 14 and on Day 28, mice were challenged with 0.05 mL of 10 mg/mL heat-killed PA suspension via intratracheal route. Then from day 28 to day 42 daily administration of DNase I inhalation at [5 microgram/50 microliters] or PBS solution [controls] per mice per day. Following the induction of granulomatous inflammation the active treated DNase 1 group demonstrated a statistically protective effect on lung inflammation.

Summary and conclusions of Inventors' proprionic bacterium animal model study number IPST 20181114-1 (90). This study aimed at evaluating the efficacy of DNase I to reduce P. Acnes-induced pulmonary sarcoidosis and its complication. This model of sarcoidosis induced a translational progression of granulomatous inflammation into pulmonary fibrosis. This is different than the idiopathic pulmonary fibrosis which the fibroblastic foci and the dense collagen is diffusely distributed in the interstitium of the lungs and brings a higher mortality rate.

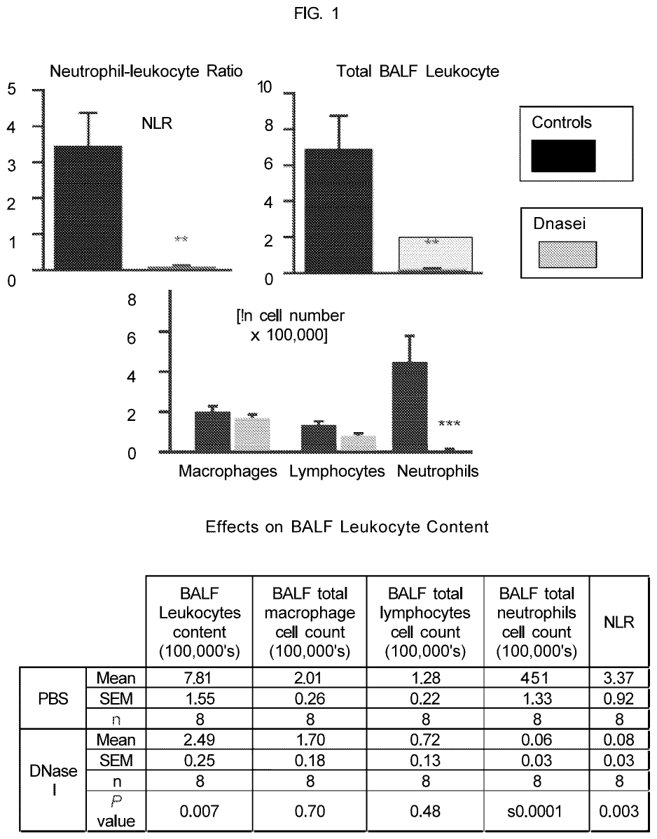

(FIG. 1) Effect of DNase 1 on bronchoalveolar lavage fluid (“BALF”) leukocytes content in sarcoidosis animal model treated with DNase 1 versus controls.

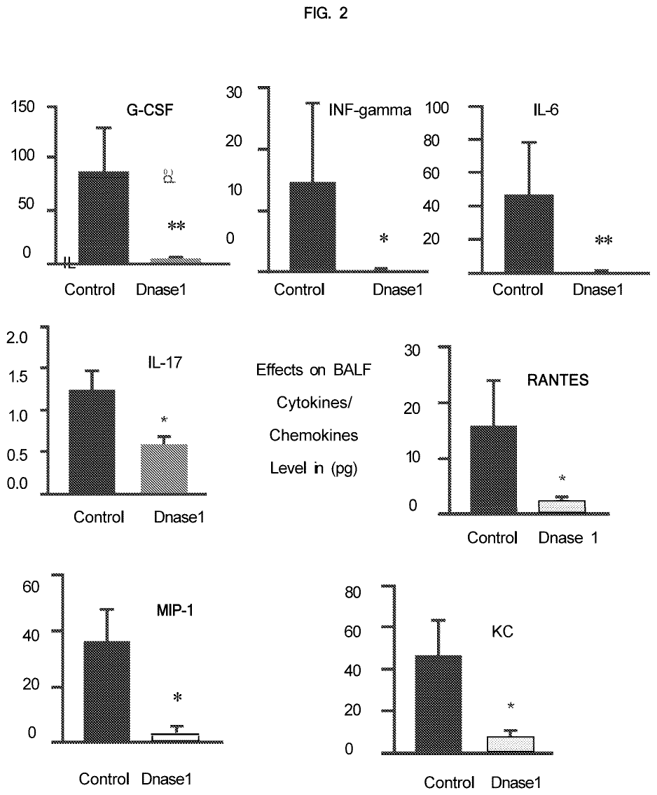

(FIG. 2) Effects of DNase 1 in BALF Cytokines/Chemokines Level in sarcoidosis animal model treated with DNase 1 versus controls.

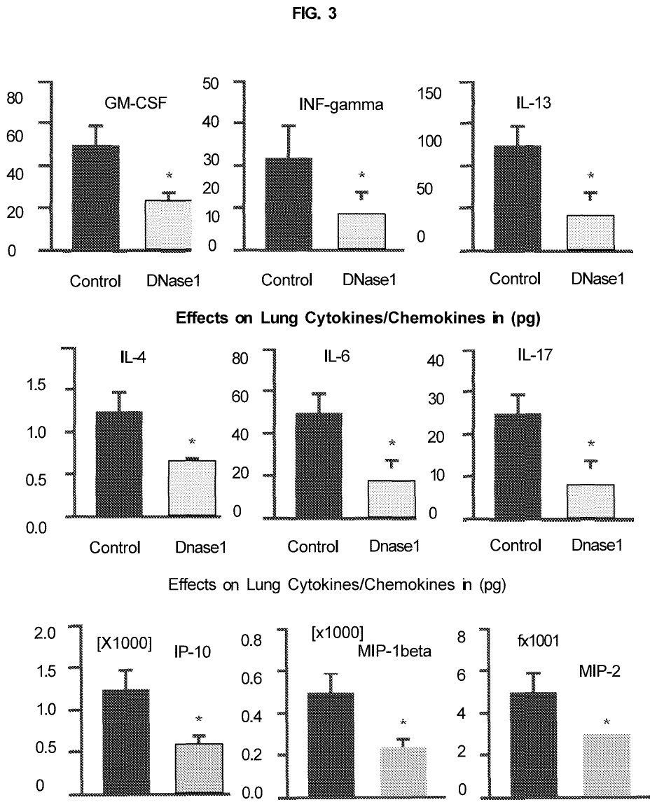

(FIG. 3) Effects of DNase 1 on Lung Cytokines/Chemokines Level in sarcoidosis animal model treated with DNase 1 versus controls.

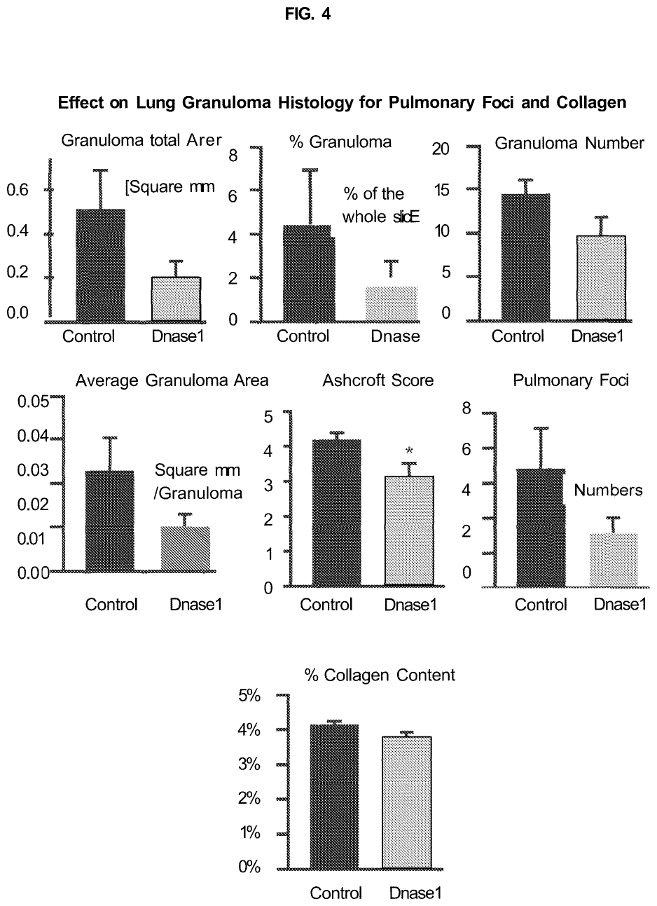

(FIG. 4) Effects of DNase 1 on Lung Granulomas Histology for Pulmonary Foci and Collagen in sarcoidosis animal model treated with DNase 1 versus controls.

In study number 20181114-1 the Inventors evaluated the effect of DNase I treatment when administered daily at a dose rate of 5 μg per mouse per day following the sarcoidosis model induction. Overall, DNase I treatment had a positive effect on lung inflammation. BALF leukocytes number and its composition such as the neutrophil to leukocyte ratio were significantly lower. BALF leukocytes content was three (3) times lower in mice receiving DNase I, when compared to vehicle treated mice (2.49×105 and 7.81×105, respectively). BALF neutrophils content was significantly lower in mice receiving DNase I (0.06×105), compared to mice receiving PBS (4.51×105). The sarcoidosis evaluation using the neutrophil to leucocyte ratio clearly demonstrated that this inflammatory marker was lower in DNase I treated mice (0.08) when compared to vehicle treated mice (3.37). (FIG. 1).

Many cytokines and chemokines (BALF and lung homogenate) were statistically significantly lower in mice receiving DNase I when compared to mice receiving PBS. 1.) In BALF, G-CSF and MIP-1 beta (also known as CCL4) which are involved in the stimulation and activation of granulocytes formation were significantly decreased in the DNase I treated mice. 2.) IFN-gamma and IL-6, which are related to macrophages, were also decreased by the DNase I treatment. 3.) RANTES, which is a chemotactic for T cells, eosinophils, and basophils, plays an active role in recruiting leukocytes into inflammatory sites. (FIG. 2 BALF) (FIG. 3 Lung) A diminution of all these cytokines in the lungs, via the DNase I treatment, may be associated in the reduction of the pulmonary fibrosis associated with sarcoidosis. The same outcomes are noted in the lung homogenate.

Altogether, these results confirm that DNase I reduced lung inflammation associated with granulomatosis. This data in direct association with inflammatory histology. Associated H&E-stained slides of the lung demonstrate a reduction of the number of granulomas and their average area, such as the total granuloma area. (FIG. 4) The effect of DNase I on granulomas' formation correlates with the lower cytokines/chemokines level in BALF and in lung homogenate. The reduced pro-inflammatory cytokine [inflammation] correlates with a lower edema formation in the DNase I treated mice (lung weight, lung index and protein content).

Fibrosis development is secondary to the inflammation process. It seems evident that treating sarcoidosis with DNase 1 reduced granulomatosis and the fibrosis development. The Inventors' study confirms that DNase I reduced the fibrosis development in this animal model. by reducing inflammatory cytokines and its associated inflammation, DNase I also reduced the collagen deposition in lung parenchyma and reduces the percentage of pulmonary foci. The Ashcroft score is the percentage (%) of pulmonary foci and the total collagen content in lung homogenate. DNase I reduced the Ashcroft score by 1 point (4.2 to 3.2). Pulmonary foci were significantly lower in DNase I treated mice, compared to PBS treated ones (2.1% and 4.6%, respectively) while total soluble collagen in lung homogenate was also lower in DNase I group compared to vehicle treated mice (3.3 versus 3.9 mg).

In conclusion, Inventors' study 20181114-1 demonstrates the efficacy of DNase I to alleviate pulmonary sarcoidosis-induced inflammation and pulmonary fibrosis. Therapeutic intervention of mice with DNase I after the induction in a proprionic bacterium model of sarcoidosis resulted in a significant reduction of lung inflammation and fibrosis development. Reduction of granulomatosis by DNase I treatment could avoid chronic inflammation leading to the lung fibrosis phase of the disease. This benefit may translate to the human sarcoidosis model where treatment with DNase 1 may reduce or eliminate the need for steroids. This outcome would be welcomed by patients and treating physicians alike due to the long-term adverse side effects of steroids.

Inventors' working MOA using DNase 1 in sarcoidosis patients further defined (FIG. 5). Deoxyribonuclease (DNase I) is a natural occurring enzyme that selectively hydrolyzes single and double stranded DNA. A recombinant human DNase I, Dornase Alfa, is produced in Chinese Hamster Ovary (CHO) cells and marketed as Pulmozyme®. It is approved only for the treatment of patients with cystic fibrosis. In patients with cystic fibrosis this product acts primarily as a mucolytic. However, evidence suggests that it also acts on bacterial DNA to reduce recurrent pseudomonas infections from bacteria found in a biofilm within a patient's lung (88).

Alidornase Alpha, by Protalix Biotherapeutics, is a recombinant human DNase I [nuclease] with a molecular weight of 29,300 Daltons, expressed in plant cells with a chemical modification of the enzyme that imparts improved resistance to actin inhibition, thereby giving Alidornase Alpha a much longer duration of nuclease activity. This enzyme is like dornase alfa (Pulmozyme), which is also used to treat cystic fibrosis (CF) by reducing mucus viscosity. Alidornase alpha is being investigated for its potential benefits and effectiveness in treating CF and other conditions involving thick mucus secretions. The drug has completed an abbreviated Phase 2 study in older cystic fibrosis patients in Israel.

In a mouse model of pulmonary sarcoidosis the Inventors using the bacterium Propionibacterium acnes recommended by FDA (89). Inhaled recombinant human DNase I was shown to markedly attenuate the inflammatory pathological processes leading to granuloma formation. (IPST Study Number: 20181114-1) (90). This model mimics the human pathological process of pulmonary sarcoidosis through initiation of granuloma formation via a bacterium found in lungs of patients with pulmonary sarcoidosis as outlined above. A complex of protein and bacterial DNA may be the initial inciting antigen that triggers the subsequent immunological cascade leading to granuloma formation (91). The DNase I activity of the inhaled enzyme is postulated to reduce or eliminate the antigenic complex and thus inhibit granuloma formation.

By extension through the same mechanism of DNase activity is expected to degrade the microbial DNA within the protein/DNA complex present in lungs of patients with pulmonary sarcoidosis. The reduction of the burden of bacterial DNA could then reduce or eliminate the antigen responsible for the persistent immunological stimulation causing granuloma formation. Of note, a subset of pulmonary sarcoidosis patients had elevated levels of beta-actin in bronchoalveolar lavage fluid, which may act as an antigen to increase the risk of developing pulmonary fibrosis (95).

The actin inhibition resistant of Alidornase Alpha may offer longer duration of efficacy for sarcoidosis patients with increased levels of intrapulmonary actin. In addition, the Protalix Biotherapeutics drug has mucolytic activity on sputum containing extensive DNA from either bacterial or human cell origin in cystic fibrosis. The drug would improve clearance of thick sputum if occurring in patients with pulmonary sarcoidosis. Most sarcoid patients have a dry nonproductive airway.

BRIEF SUMMARY OF THE INVENTION The present invention is directed to methods for treating pulmonary sarcoidosis comprising the therapeutic administration of DNase1 or other [nuclease enzymes] for the breakdown of excessive free lung microbial and mitochondrial DNA unassociated with mucous [cystic fibrosis]. Such free DNA enters dendritic cells and macrophages by way of toll receptor 2 (TLR2) to stimulate toll receptor 9 (TLR9) and initiate the inflammation cascade associated with sarcoidosis. Therapeutic DNase 1 in sarcoidosis lungs reduces DNA levels and associated stimulation of cytokines, inflammation, and granulomas. When therapy is indicated, corticosteroids (CS) are usually the first-choice drugs, because they work more reliably and more rapidly than all other alterative agents (106) (107). The Inventors' mechanism of action will allow for less steroid use in patients with sarcoidosis and the associated high complication rate. The treatment proposed would treat the cause of the disease. Thus, reducing or eliminating the need for further anti-immune therapies with the seriously associated side effects. Further, certainly reducing the amount and duration of such toxic therapies. There is a significant medical need for new safe drugs to treat sarcoidosis. The DNase 1 therapy has been available for cystic fibrosis for years with an acceptable safety profile.

DETAILED DESCRIPTION OF THE INVENTION (FIG. 5)—Detailed description of the Inventions Mechanism of Action. Abbreviations: Ab=antibody; Ag=antigen; AmyLP=amyloid protein; hDNA=human DNA; mDNA=Mitochondrial DNA; IFN=interferon; IFN gamma; IL12=interleulin-12; IL18=interleukin-18; IL6=interleukin-6; MHC=major histocompatibility complex; NFKB=nuclear factor-kB; PAMPS=pathogen-associated molecular patterns; SAA=serum amyloid A; TCR=T cell receptor; Th1=Type 1 helper T cell; Th17=Type 17 helper T cell; Th17.1=IFN-γ-producing Th17 cells; TLR2=toll-like receptor 2; TLR9=toll-like receptor 9; TNF=tumor necrosis factor; mTORC1=The mechanistic target of rapamycin; Treg=regulatory T-cells.

There is evidence of the presence of microbial DNA from mycobacteria, propionibacteria and borrelia and other bacteria in sarcoidosis tissues suggesting they may participate in sarcoidosis granuloma formation (92/96). Mitochondrial DNA also contributes to the free DNA load. (FIG. 5).

Mitochondrial DNA (mtDNA) is a small, circular chromosome found inside the mitochondria, which are the energy-producing organelles in eukaryotic cells. The endosymbiotic theory suggests that mitochondria originated from free-living bacteria that were engulfed by ancestral eukaryotic cells, leading to a symbiotic relationship. Both bacterial DNA and mitochondrial DNA are circular in shape, unlike the linear DNA found in the nuclei of eukaryotic cells. Mitochondria are cellular organelles that orchestrate a vast range of biological processes, from energy production and metabolism to cell death and inflammation. Despite this seemingly symbiotic relationship, mitochondria harbor within them a potent agonist of innate immunity: their own genome. Release of mtDNA into the cytoplasm and out into the extracellular milieu activates a plethora of different pattern recognition receptors and innate immune responses, including cGAS-STING, TLR9 and inflammasome formation leading to, among others, robust type I interferon responses. mtDNA can be released from mitochondria, the various inflammatory pathways triggered by mtDNA release and its myriad biological consequences for health and disease (97). (FIG. 5)

Bacterial amyloid curli acts as a carrier for such DNA to elicit an autoimmune response (93) (94) (FIG. 5).

Serum amyloid A derived from the host participates in the granuloma formation by regulating inflammation through Toll-like receptor-2 (TLR2) (99). Serum amyloid A is localized to macrophages and giant cells within sarcoidosis granulomas and correlates with CD3 (+) lymphocytes, linking expression to local Th1 responses (96). Serum amyloid A activated NF-kappaβ in TLR2-expressing human cell lines, regulated experimental Th1-mediated granulomatous inflammation through interferon (IFN)-gamma, tumor necrosis factor, interleukin (IL)-10, and TLR-2. It also stimulates production of tumor necrosis factor, IL-10, and IL-18 in lung cells from patients with sarcoidosis which is inhibited by blocking TLR-2 (96). (FIG. 5).

mTORC1 (mechanistic target of rapamycin complex 1) is indeed associated with inflammation. It plays a significant role in regulating immune responses and inflammation by influencing the function and metabolism of various immune cells. For instance, mTORC1 signaling is crucial for the differentiation and function of proinflammatory macrophages (M1 macrophages), which are involved in the body's inflammatory response. Additionally, mTORC1 can be activated by pro-inflammatory cytokines, such as TNF-α, which further promotes inflammatory processes. Dysregulation of mTORC1 signaling has been linked to various inflammatory diseases and conditions, making it a potential target for therapeutic interventions aimed at controlling inflammation. (98). Immunologically, mTOR is a critical regulator of immune function through integrating numerous signals from the immune microenvironment, which coordinates the functions of immune cells and T cell fate decisions. Recently the crucial role of mTOR in immune responses is dramatically more appreciated (99.) With the greater appreciation of cellular metabolism as an important regulator of immune cell function, mTOR is proving to be a vital link between immune function and metabolism. In this review, we discuss the ability of mTOR to direct the adaptive immune response. Specifically, we focus on the role of mTOR in promoting differentiation, activation, and function in T cells, B cells, and antigen-presenting cells (100). (FIG. 5)

### DRAWINGS

(FIG. 1) Effect of DNase 1 on bronchoalveolar lavage fluid (“BALF”) leukocytes content in sarcoidosis animal model treated with DNase 1 versus controls.

(FIG. 2) Effects of DNase 1 in BALF Cytokines/Chemokines Level in sarcoidosis animal model treated with DNase 1 versus controls.

(FIG. 3) Effects of DNase 1 on Lung Cytokines/Chemokines Level in sarcoidosis animal model treated with DNase 1 versus controls.

(FIG. 4) Effects of DNase 1 on Lung Granulomas Histology for Pulmonary Foci and Collagen in sarcoidosis animal model treated with DNase 1 versus controls.

(FIG. 5)—Detailed drawing of the Inventions Mechanism Of Action with excess mitochondrial, microbial, and other free DNA stimulating Toll Receptor 9 (TLR 9) that causes inflammation and granuloma formation.

1. A method of treating pulmonary sarcoidosis by administrating to a patient in need of a therapeutic effective dose of a DNA [endo] nuclease to clear excess microbial, mitochondrial, and free DNA present in such patients' lungs.

2. The method of embodiment 1 where the nuclease is a recombinant human DNase 1 (Dornase Alpha).

3. The method of embodiment 1 where the nuclease is a plant derived recombinant human DNase 1 (Alidornase Alpha) where DNase 1 was engineered to resist actin degradation.

4. The method of embodiment 1 where the nuclease is one or more of the following from the family of DNase 1 variants of DNase 1 L 1, DNase 1 L2, DNase 1 L3 or combination thereof.

5. The method of embodiment 1 where the nuclease is inhaled by a Nebulizer which converts liquid medication into a fine mist, which is then inhaled through a mouthpiece or mask.

6. The method of embodiment 1 where the nuclease is inhaled by a Soft Mist Inhaler which produces slow-moving mist that allows for easier inhalation and better deposition of the medications in the lungs.

7. The method of embodiment 1 where the nuclease is inhaled by a Dry Powder Inhaler which delivers medication in a dry powder form deeply into the lungs.

8. The method of embodiment 1 where the nuclease is inhaled by a Metered-Dose Inhaler which delivers medication in a dry powder form which is inhaled into their lungs.

9. The method of embodiment 1 in which the pulmonary sarcoidosis patient is on steroids which may be tapered.

10. The method of embodiment 1 in which the pulmonary sarcoidosis patient is having an acute exacerbation of their disease.

11. The method of embodiment 1 in which the pulmonary sarcoidosis patient is stable and needs a maintenance dose to prevent active exacerbations.

12. The method of embodiment 1 in which the pulmonary sarcoidosis patient is being treated in combination with an intravenous dose of a DNA nuclease.

13. The method of embodiment 12 in which the DNase nuclease is a recombinant human DNA 1 nuclease.

14. The method of embodiment 12 in which the □Nase nuclease is a recombinant human DNA 1 nuclease with biologic half-life extensions.
